(19) United States
(12) Patent Application Publication
(10) Pub. No.: US 2022/0118205 A1
(43) Pub. Date: Apr. 21, 2022

(54) VENTILATION TUBES WITH INFLATABLE CUFFS

(71) Applicant: Airway Medix S.A., Warsaw (PL)

(72) Inventor: Oron ZACHAR, Tel Aviv (IL)

(21) Appl. No.: 17/275,149

(22) PCT Filed: Sep. 25, 2019
(86) PCT No.: PCT/IB19/01033

§ 371 Date: Mar. 10, 2021

Related U.S. Application Data
(60) Provisional application No. 62/735,875, filed on Sep. 25, 2018.
(60) Provisional application No. 62/775,375, filed on Dec. 4, 2018.

(51) Int. Cl. A61M 16/04

(52) U.S. Cl. CPC A61M 16/0443

(57) ABSTRACT

Embodiments of the invention relate to a ventilation device comprising: a. a ventilation tube having proximal and distal ends; and b. an inflatable cuff having a proximal bulge portion and a distal neck portion disposed distal to the bulge portion, the inflatable cuff being mounted around the ventilation tube to define proximal and distal cuff attachment locations which are both fixed on an outer surface of the ventilation tube.

### FIELD

The present invention relates, generally, to systems and methods for airway ventilation tube products, such as tracheal tubes and tracheostomy tube comprising inflatable cuffs.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient with artificial ventilation, a ventilation tube may be used to control the flow of air into the patient. It is desirable to provide a seal between the outside of the tube or device and the interior walls of the trachea passage in which the tube or device is inserted. For example, tracheal tubes may be used to control the flow of air or other gases through a patient's trachea. To seal these types of tracheal tubes, an inflatable cuff may be associated with these tubes. When inflated, the cuff generally expands into the surrounding trachea to seal the tracheal passage around the tube. In order to create a good seal, it was found by practitioners that the cuff pressure should commonly be higher than 20 cm H2O. Yet, in order to avoid tracheal tissue ischemia, the cuff pressure is desired to be maintained at a pressure below 30 cm H2O. Hence, proper cuff pressure is limited to a narrow range of 20-30 cm H2O. The trachea is not an even tube and therefore slight movements of the ventilation tube may shift the cuff to more constricted or more open locations within the trachea—resulting in alteration of the original setting of the balloon pressure when first inflated. Unfortunately, in the present art of ventilation cuff balloons, the clinical pressure variation due to such movements range far beyond the desired range of 20-30 cm H2O. Moreover, human anatomy varies significantly between individuals. One prior art device is disclosed in U.S. Pat. No. 9,032,957.

### SUMMARY OF EMBODIMENTS

The present invention relates, generally, to systems and methods for airway ventilation tube products, such as tracheal tubes and tracheostomy tube comprising inflatable cuffs. In particular, various embodiments are directed to efficient methods of improving the sealing properties and pressure stability of the inflated cuff when in use.

Embodiments of the invention relate to a ventilation device comprising: a. a ventilation tube having proximal and distal ends; and b. an inflatable cuff having a proximal bulge portion and a distal neck portion disposed distal to the bulge portion, the inflatable cuff being mounted around the ventilation tube to define proximal and distal cuff attachment locations which are both fixed on an outer surface of the ventilation tube.

In some embodiments, (i) when the tube-mounted cuff is deployed within a human trachea sized for the ventilation tube so that (A) the ventilation tube is co-axial with the human trachea and (B) the tube-mounted cuff is uninflated or inflated to a pressure of 5 cm H2O, a widest portion of the bulge portion of the mounted cuff is in wrinkled contact with the trachea.

In some embodiments, when the tube-mounted cuff is deployed within a human trachea sized for the ventilation tube so that (A) the ventilation tube is co-axial with the human trachea and (B) the tube-mounted cuff is uninflated or inflated to a pressure of 25 cm H2O, a widest portion of the bulge portion of the mounted cuff is in wrinkled contact with the trachea.

### BRIEF DESCRIPTION OF THE DRAWINGS

A presently preferred embodiment of the invention will be described in detail, in conjunction with the accompanying drawings, in which:

FIGS. 1A-1B, 2A-2E, 3A-3B, 4A-4B and 6A-6B illustrate an exemplary ventilation device of component(s) thereof.

FIG. 5 illustrates contact length as a function of pressure.

FIGS. 7A-7B and 8A-8B relate to experiments performed on an exemplary cuff.

FIG. 9 illustrates a relationship between a volume parameter and a pressure parameter.

FIGS. 10A-10C illustrate tables and graphs which exemplify the relationship between selected ventilation tube sizes and associated human trachea sizes characterized by their diameter dimension.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—any combination of features can be included in any embodiment and/or omitted from any embodiments.

There is a need for ventilation tubes with inflatable cuff that have improved pressure stability and/or good contact even at low pressure (e.g. to handle the fact that the trachea is not a perfect cylinder). Embodiments of the present invention relate to apparatus and methods for achieving a relatively stable cuff pressure, within the range of 20-30 cm H2O, under varying volume on the order of 10% of the cuff volume. Tracheal tubes cuff volume within the trachea is about 5 cc to 10 cc and tracheostomy tube cuff volumes are even smaller 3 cc to 5 cc. Previously known art of ventilation tube cuffs, a volume difference of 0.4 cc already takes the cuff pressure significantly out of range.

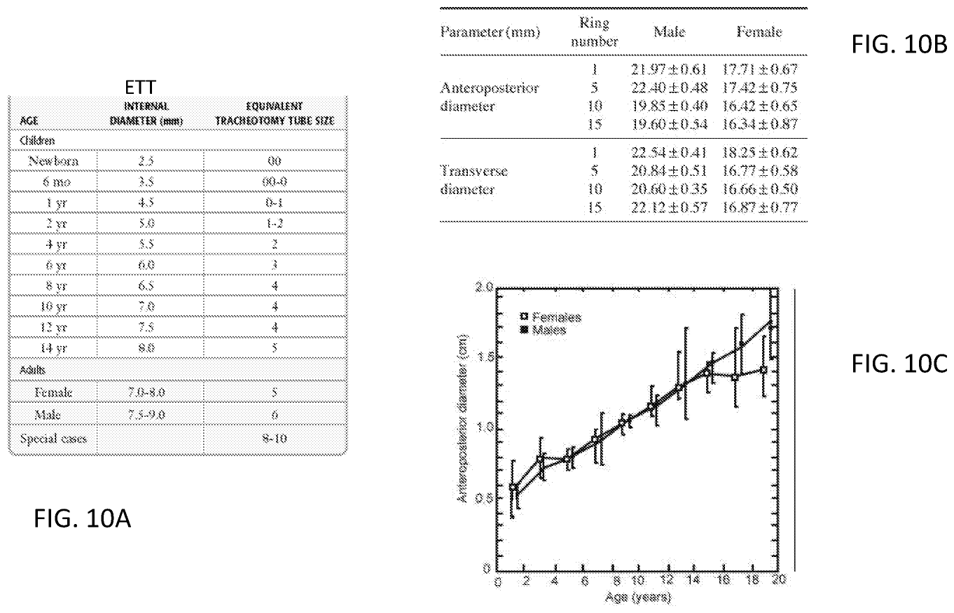

The present invention introduces ventilation tubes with new cuffs purposefully engineered to have a pressure/volume inflation curve that is providing for unprecedented pressure stability when inflated within a human trachea. Unlike previously known art of ventilation tube cuffs, the present invention introduces cuffs that behave significantly different when inflated within an enclosing tube compared with free space inflation. The cuffs pressure curve is different when inflated within different tube diameters. Ventilation tubes and associated cuffs are sized according to intended human user, as summarized in the tables shown in FIGS. 810 to 10C. In particular, the cuffs size, shape, and elastic properties are configured to achieve the specific desired safe pressure range of 20-30 cm H2O when inflated within an enclosing tube of similar dimensions to the associated human trachea.

It helps to highlight from the outset certain distinguishing feature of the present invention embodiments in comparison with known art. For ease of reference the following numbers in the figures are meant to refer to as follows
100—ventilation device comprising a ventilation tube 106 and an inflatable cuff 200
106—catheter tube or ventilation tube (e.g. ETT or tracheostomy tube)
102—proximal end of ventilation tube 106
107—distal end of ventilation tube 106
130—central axis of ventilation tube 106
200—inflatable cuff (e.g. balloon cuff) mounted around the ventilation tube 106
211—proximal cuff attachment location of cuff 200 on outer surface of tube 106
212—distal cuff attachment location of cuff on outer surface of tube 106
203—cuff balloon wall
104—inflation lumen for inflating the inflatable cuff 200
109—proximal inflation inlet of inflation lumen 104
105—distal inflation inlet into cuff 200
287—proximal bulge portion of cuff 200
289—distal neck portion of cuff 200
901—proximal direction defined according to proximal end 102 of the ventilation tube 106
951—distal direction defined according to distal end 107 of the ventilation tube 106
108—enclosing tube which is either (i) a human trachea that is sized for the ventilation tube 106 to which cuff 200 is mounted; and/or (ii) an external ‘in vitro’ rigid straight (i.e. perfectly cylindrical) test tube whose interior width matches that of a human trachea that is ‘sized for’ the ventilation tube 106 to which cuff 200 is mounted and/or an in vitro' rigid straight (i.e. perfectly cylindrical) test tube whose interior width/diameter matches is between 18 mm and 22 mm (e.g. between 18 mm and 21 mm or between 18 mm and 20).

For the present disclosure and as shown in all drawings, it is assumed that when ventilation tube 106 is in enclosing tube 108 (i.e. either a human trachea or an in-vitro enclosing tube), the ventilation tube is co-axial with the enclosing tube 108. For the present disclosure, any feature disclosed with respect to a human trachea (e.g. a trachea that is ‘sized for’ tube 106) may also be provided with respect to an external ‘in vitro’ rigid straight enclosing (i.e. perfectly cylindrical) test tube whose interior width matches that of a human trachea that is ‘sized for’ the ventilation tube 106 to which cuff 200 is mounted and/or an in vitro' rigid straight (i.e. perfectly cylindrical) test tube whose interior width/diameter is between 18 mm and 22 mm (e.g. between 18 mm and 21 mm or between 18 mm and 20 mm).

Any reference to ‘hyper-elastic material’ may also relate to ‘elastic’ material as well.

Inflation of a cuff without specifying conditions refers to (i) inflation with air and (ii) at ambient conditions (i.e. standard room conditions of 20 degrees and one atmosphere ambient pressure).

Whenever a cuff is in free space, this is understood to require that the cuff is not disposed in any trachea or any enclosing tube.

The length of the cuff LENGTH_{CUFF} is the longitudinal displacement between the proximal 211 and distal 212 cuff attachment locations. Generally, the LENGTH_{CUFF} is independent of inflation conditions.

An X% lengthwise portion (i.e. segment) of the cuff is a portion of the cuff whose length is X% of LENGTH_{CUFF}. Any longitudinal cut of the cuff may be specified by as a X% lengthwise portion of the cuff by specifying (i) a value of X ; and (ii) a center longitudinal-location on the ventilation tube around with the X% lengthwise portion of the cuff is longitudinally centered.

A X% length fraction of the cuff is a portion of the cuff whose length (i.e. along the central axis of the tube) is X% of the length of the cuff.

A most distal X% of the cuff is a X% length fraction of the cuff which is distally bound by the distal 212 cuff attachment location. a most distal X% of the cuff is a X% length fraction of the cuff which is proximally bound by the proximal 212 cuff attachment location.

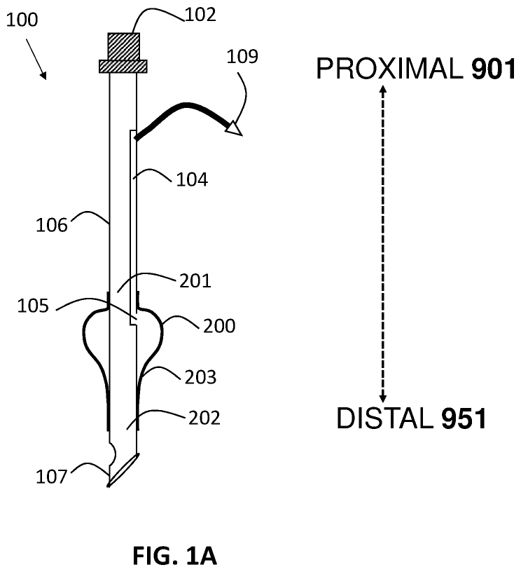

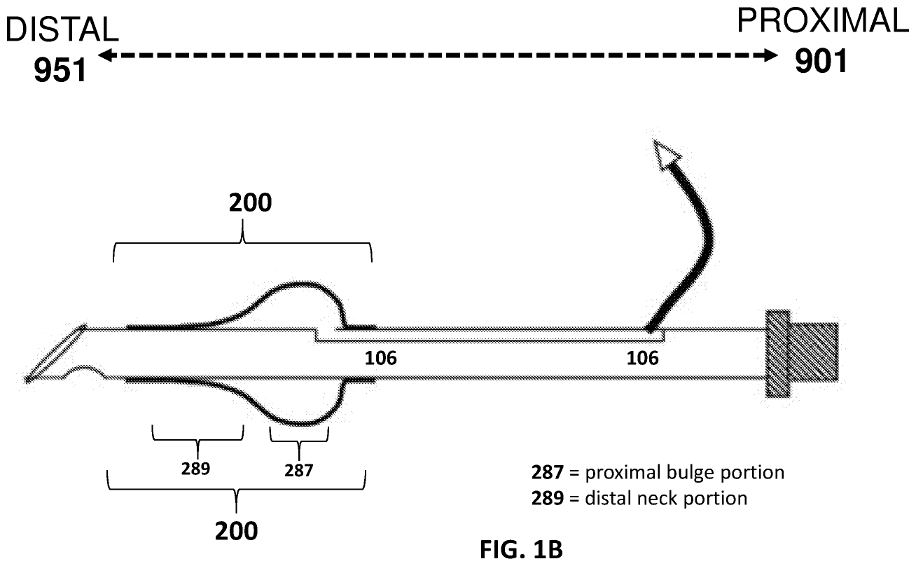

### FIGS. 1A-1B

FIGS. 1A-1B illustrate an example ventilation device 100 comprising (A) a ventilation tube 106 (i.e. typically an endo-tracheal type (ETT) or a tracheostomy tube) and aninflatable cuff 200 mounted thereto. The ventilation tube has proximal 102 and distal 107 ends which define the proximal 901 and distal 951 directions shown in FIG. 1.

As shown in FIGS. 1A-1B, cuff 200 has a proximal bulge portion 287 and a distal neck portion 289 disposed distal to the bulge portion. As will be discussed below, one salient feature provided by embodiments of the invention is the deformation of the distal neck portion as gas (e.g. air) is forced into cuff 200.

The ventilation tube 106 is intended for use within a human trachea of a particular size according to requirements of the medical community—i.e. the ventilation tube 106 is intended for use in a human trachea that is ‘sized for’ the ventilation use.

When the tube-mounted cuff 200 is (i) deployed within a human trachea sized for the ventilation tube (i.e. so a portion of the ventilation tube 106, around which cuff 200 is mounted, is co-axial with the human trachea) and (ii) is uninflated or inflated to a pressure of 5 cm H2O, a widest portion of the bulge portion 287 of the mounted cuff is in wrinkled contact with the trachea.

Thus, one salient feature of ventilation device 100 is that there is no need to ‘stretch’ (i.e. via inflation) the ventilation tube into contact with the human trachea sized for ventilation tube 106. Thus, even at low pressures (e.g. at 5 cm H2O or even less) cuff 200 has sufficient volume to contact the inner wall of the human trachea—in this sense, ventilation device 100 may be said to belong to a class of devices informally known as high volume low pressure devices.

Typically, the mounted cuff 200 is disposed, adhesively or otherwise, towards the distal end 107 of the endotracheal tube 106. The cuff 200 may be inflated and deflated through a proximal inflation inlet 109 via a lumen 104 in fluid communication with the cuff 200, typically through a distal inflation inlet hole 105 in the inflation lumen 104.

The cuff 200 has a proximal opening 201 and a distal opening 202 formed in the cuff walls 204 sized to accommodate the endotracheal tube 106. The proximal opening 201, located closer to the “ventilation machine end” 102 of the tube 106, and a distal opening 202, located closer to the “patient end” 107 of the tube 106, are typically used to mount the cuff 200 to the tube 106.

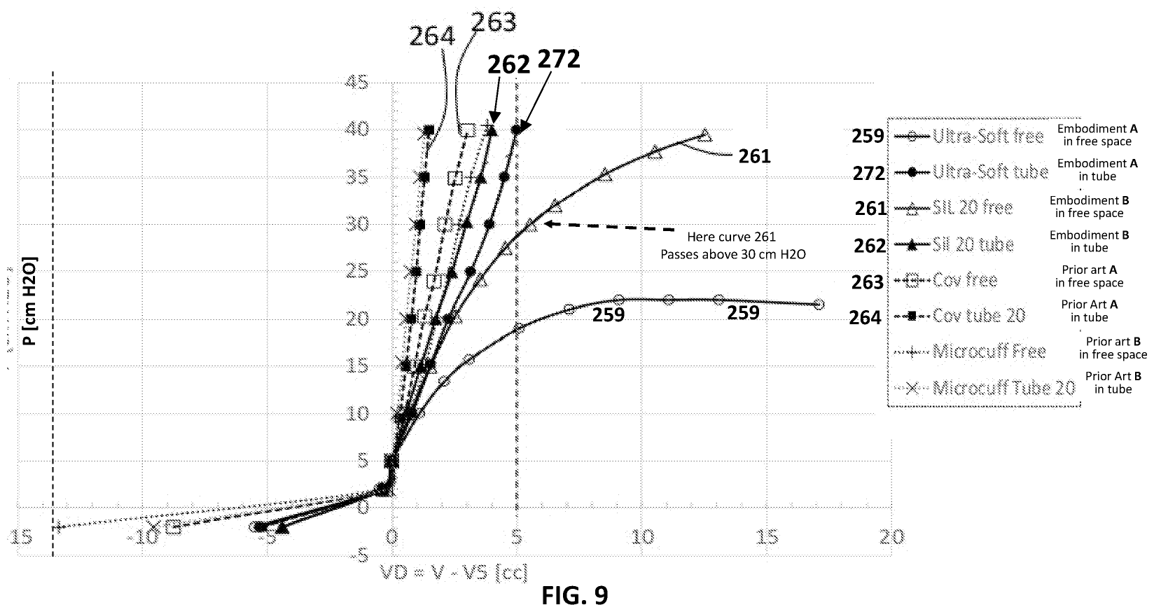

Material—In different embodiments, cuff 200 is constructed of a relatively soft material. One example inflatable cuff 200 is referred to as SIL 20. This cuff 200 (i.e. SIL 20) is constructed of silicone, has a Shore A hardness of about A20, and a thickness of between 0.2 mm and 0.4 mm—e.g. 0.2 mm A discussion of some properties of SIL 20 is provided below—see, for example, curves 261, 262 of FIG. 9.

Another example inflatable cuff 200 is referred to as Ultra-Soft and is constructed of thermoplastic elastomer (TPE) having a Shore OO hardness of OO38 and a thickness of between 0.4 mm and 0.7 mm—e.g. about 0.4 mm A discussion of some properties of Ultra-Soft is provided below—see, for example, curves 259, 272 of FIG. 9.

In different embodiments, the material and/or thickness of the material from which cuff 200 is constructed provides sufficient deformability and/or elasticity to provide one or more features (e.g. related to stretching) disclosed herein.

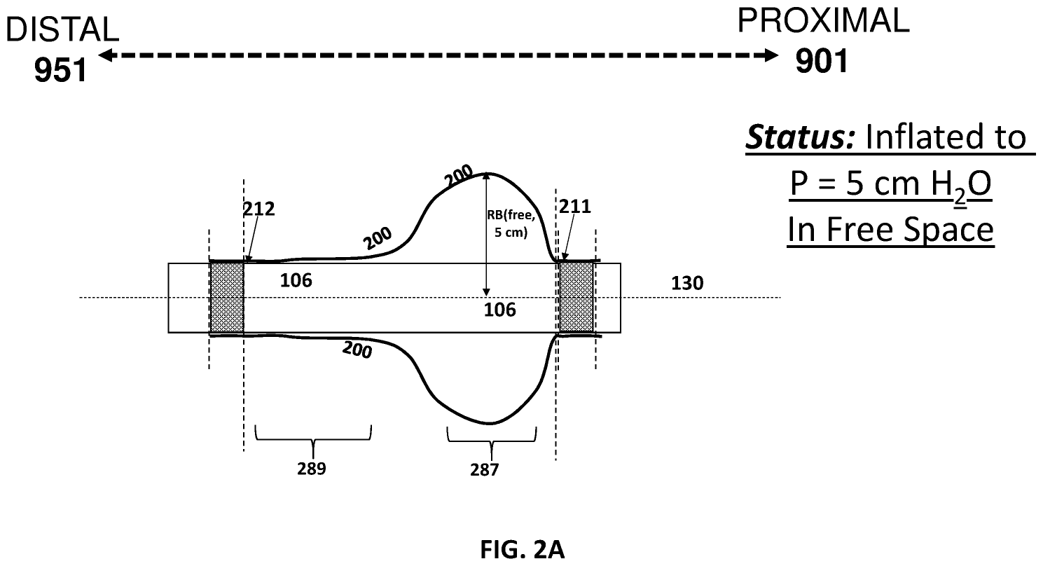

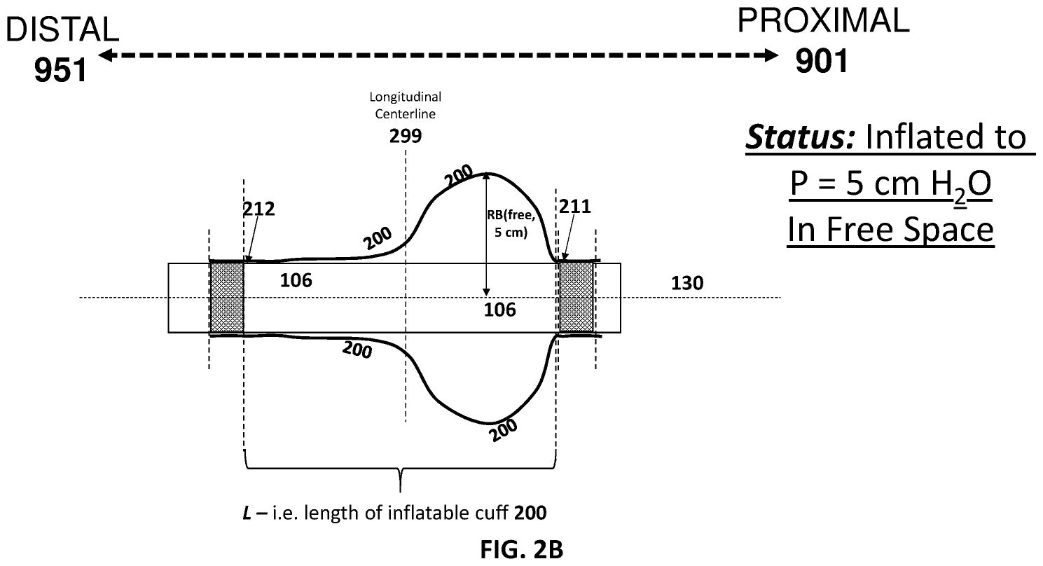

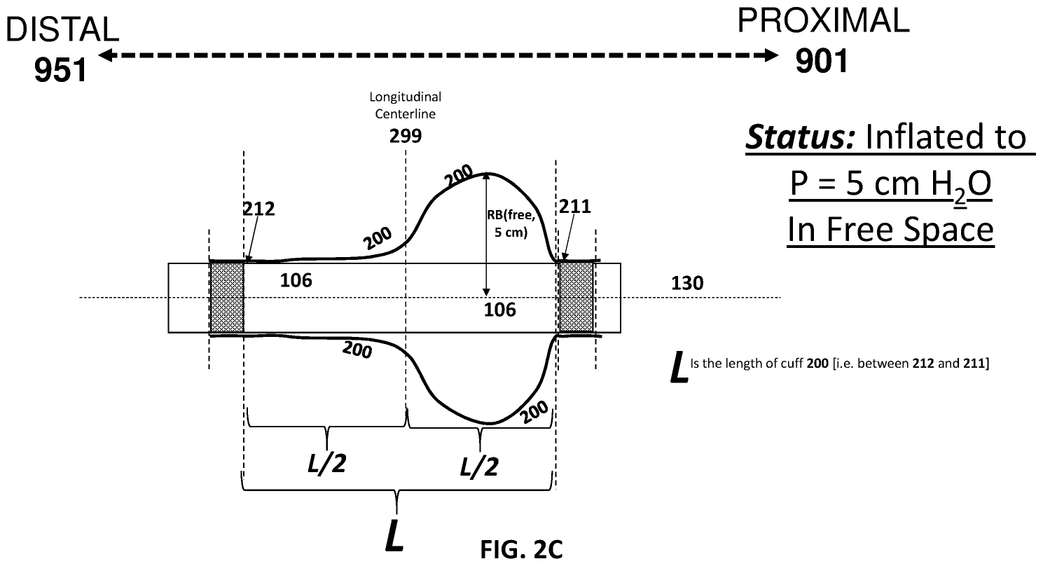

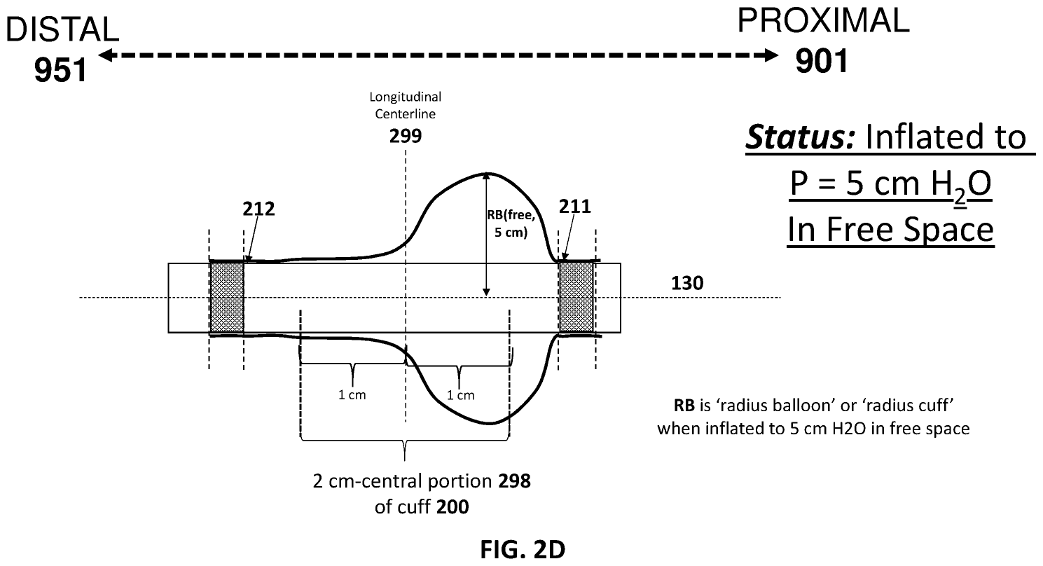

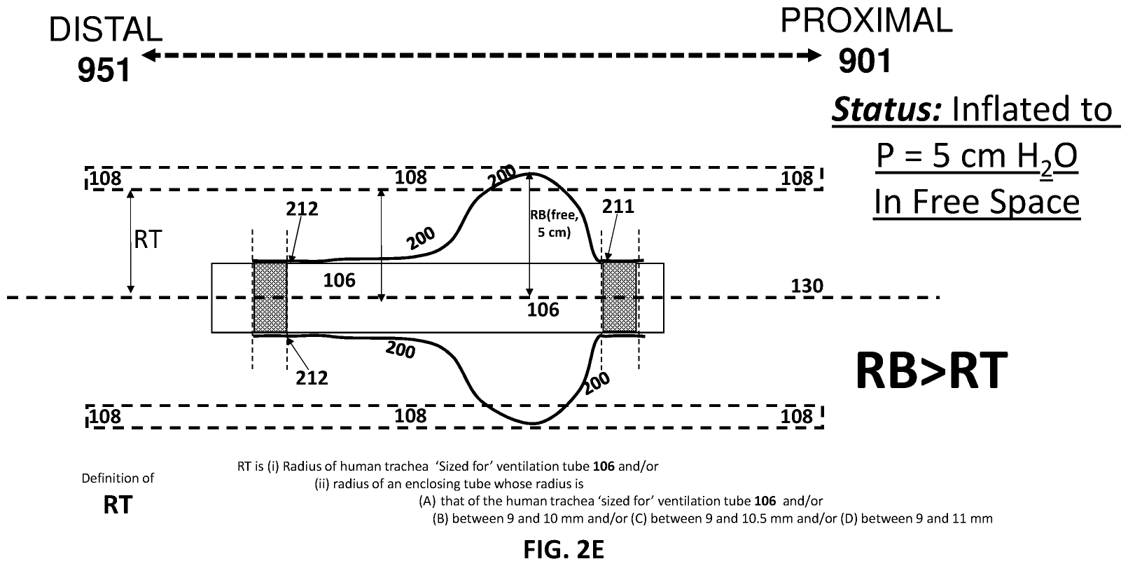

FIG. 2A illustrates cuff 200 in free space when inflated to 5 cm H2O of pressure. The radius of the cuff is shown as RB(free, 5 cm) where RB is the largest radius of the cuff 200 (e.g. balloon cuff 200) in bulge 287 portion thereof. Also shown in FIG. 2A are: (i) 211—proximal cuff attachment location of cuff 200 on outer surface of tube 106; (ii) 212—distal cuff attachment location of cuff on outer surface of tube 106; and (iii) 130—central axis of ventilation tube 106.

As shown in FIGS. 2B-2D, the ‘length’ inflatable cuff (L or LENGTH_{CUFF}) is the longitudinal displacement between the proximal 211 and distal 212 cuff attachment locations. Generally, the length is independent of inflation conditions. Also illustrated in FIG. 2B is the longitudinal centerline 299, which is disposed longitudinally halfway between proximal 211 and distal 212 cuff attachment locations. FIG. 2D shows the 2 cm-central portion 298 of cuff 200.

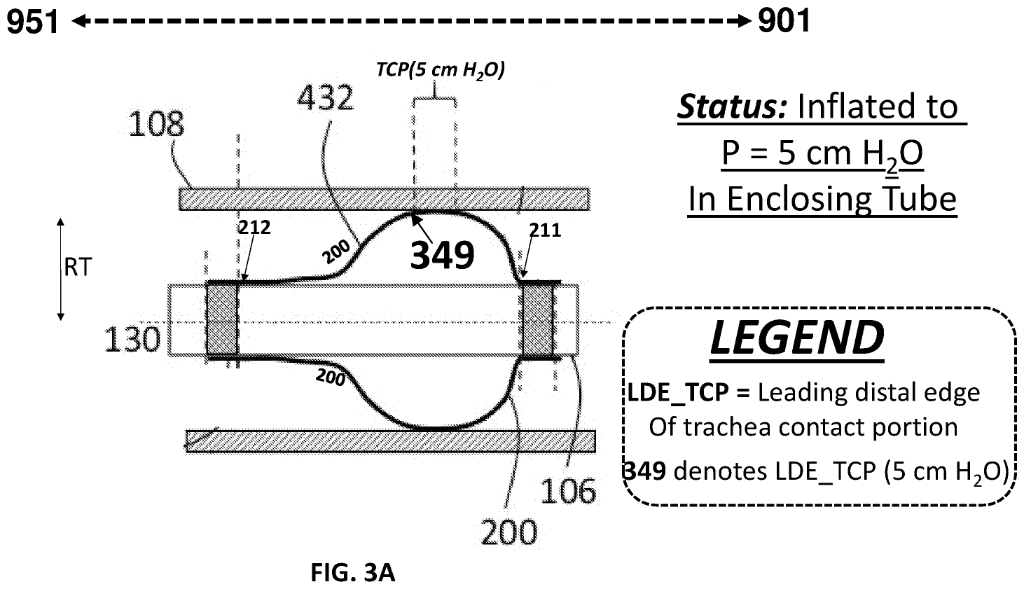

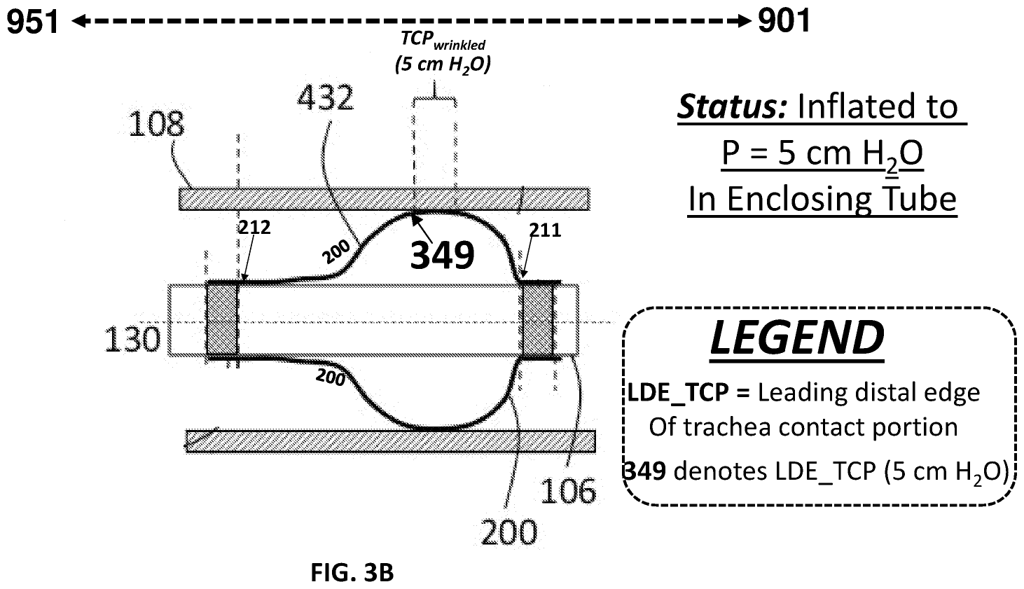

FIG. 3A illustrates the same cuff 200 of FIGS. 2A-2E. In FIGS. 2A-2E the cuff 200 is in free space. In FIG. 3A, the cuff 200 is disposed within enclosing tube 108 which is either (i) a human trachea that is sized for the ventilation tube 106 to which cuff 200 is mounted; and /or (ii) an external ‘in vitro’ rigid straight (i.e. perfectly cylindrical) test tube whose interior width matches that of a human trachea that is ‘sized for’ the ventilation tube 106 to which cuff 200 is mounted and/or an in vitro' rigid straight (i.e. perfectly cylindrical) test tube whose interior width/diameter matches is between 18 mm and 22 mm (e.g. between 18 mm and 21 mm or between 18 mm and 20 mm). In FIGS. 3A-3B, the cuff 200 (i.e. which is disposed within enclosing tube 108) is inflated to a pressure of 5 cm H2O.

The reader is invited to compare FIG. 2A to FIG. 3A. FIG. 2A shows RB(free, 5 cm) where RB is the largest radius of the cuff 200 (e.g. balloon cuff 200) in bulge 287 portion thereof. However, the radius of enclosing tube 108 is less than RB(free, 5 cm). Thus, when cuff 200 is inflated to a pressure of 5 cm H2O, there is contact between cuff 200 (e.g. in a bulge portion of cuff 200) and enclosing tube 108.

TCP stands for “Tube Contact Portion” where the “Tube” of TCB is enclosing tube 108. TCP refers to a longitudinal potion of cuff 200 that is in contact with enclosing tube 108 under specified conditions. The portion of TCP that is in wrinkled contact with enclosing tube 108 under specified conditions is TCP_{WRINKLED}.

FIG. 3A shows the TCP which is the tube contact portion of cuff 200 when (i) cuff 200 is disposed within enclosing tube 108 and (ii) cuff 200 is inflated to a pressure of 5 cm H₂O. In different embodiments, at least 10% or at least 20% or at least 50% or at least 75% of TCP(5 cm H₂O) is characterized by a presence of wrinkles—i.e. wrinkled contact between cuff 200 and enclosing tube 108. In the particular example of FIG. 3B, 100% of TCP is in wrinkled contact—i.e. the lengths of TCP(5 cm H₂O) and TCP_{WRINKLED}(LED (5 cm H₂O) are identical. This is not a requirement.

The reader is invited to compare FIG. 3A to FIG. 2E. In FIG. 2E cuff 200 is shown in free space where the location of enclosing tube 108 is shown in ghost or broken lines (i.e. it is not present in FIG. 2E). Clearly, RB>RT.

Reference is made, once again to FIG. 3A. The most distal location of TCP(5 cm H₂O) is labelled as 349 and this is known as the leading distal edge (LDE) of TCP(5 cm H₂O) and is labelled as LDE_TCP(5 cm H₂O). NOTE—once LDE_TCP(5 cm H2O) is defined by cuff 200 within the enclosing tube 108 (i.e. a human trachea 108 sized for ventilation tube 106 or any in vitro tube disclosed herein), it is noted that LDE_TCP(5 cm H2O) 349 is a property of cuff 200 (i.e. for an appropriate enclosing tube 108) and has a longitudinal location that is fixed relative to 211 and 212.

For the situation where cuff 200 is within enclosing tube 108 and inflated to a pressure of 5 cm H2O, the contour of cuff 200 in locations (e.g. locations of neck portion 289 of cuff 200) distal to labelled as 432. Cuff counter 432 is particular for the cuff pressure of 5 cm H2O and is shown in FIGS. 3A-3B.

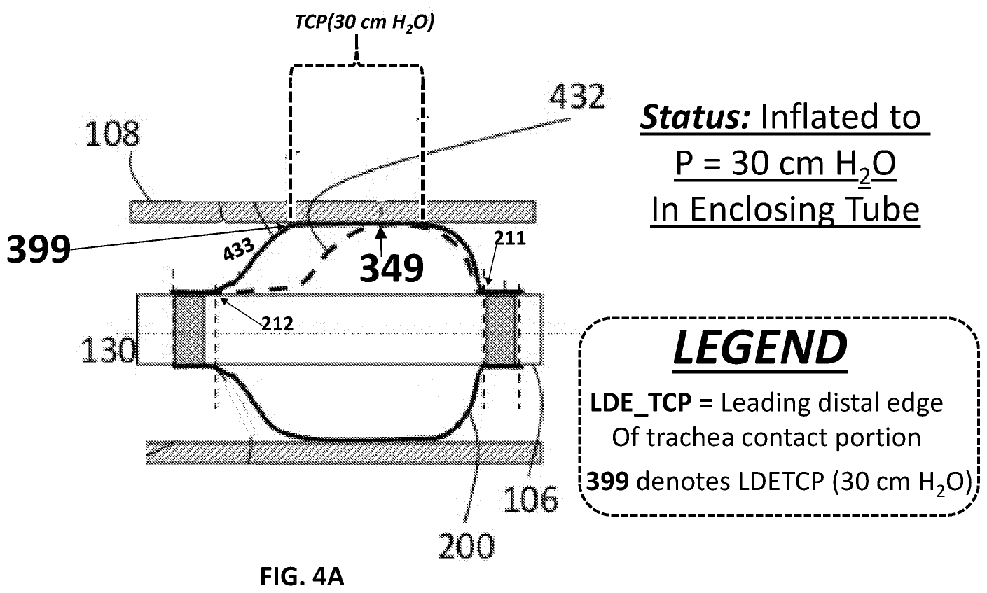

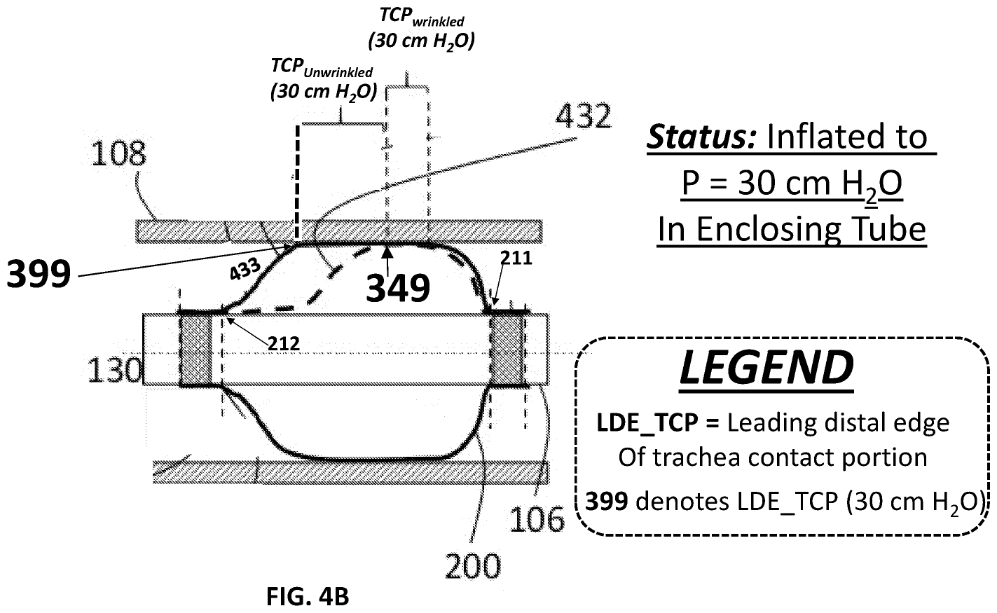

FIGS. 3A-3B versus FIGS. 4A-4B: In FIGS. 3A-3B, the cuff 200 (i.e. which is disposed within enclosing tube 108) is inflated to a pressure of 5 cm of H2O. In FIGS. 4A-4B, the cuff 200 (i.e. which is disposed within enclosing tube 108) is inflated to a pressure of 30 cm of H2O.

Comparing FIGS. 3A-3B to FIGS. 4A-4B, it is possible to make the following observations:

(i) in both FIGS. 3A-3B and FIGS. 4A-4B, at least a portion of cuff 200 is in contact with enclosing tube 108. When cuff is inflated to 5 cm of H₂O, the portion of cuff 200 in contact with enclosing tube 108 is shown as TCP(5 cm H2O) in FIG. 3A. When cuff is inflated to 30 cm of H₂O, the portion of cuff 200 in contact with enclosing tube 108 is shown as TCP(30 cm H₂O) in FIG. 4A.

(ii) Clearly LENGTH(TCP(30 cm H₂O))>LENGTH(TCP(5 cm H₂O)). This is because inflation of cuff 200 deforms the cuff 200, in particular in neck portion 289 thereof. For example, cuff 300 is constructed of material having specific elasticity and thickness.

(iii) leading distal edge of TCP(5 cm H2O) is LDE_TCP(5 cm H₂O) is labelled as element 349. Leading distal edge of TCP(30 cm H₂O) is LDE_TCP(30 cm H₂O) is labelled as element 399—because LENGTH(TCP(30 cm H₂O))>LENGTH(TCP(5 cm H₂O)), element 399 is located distal to element 349. Similar to LDE_TCP(5 cm H₂O) 349, is LDE_TCP(30 cm H₂O) 399 a property of cuff 200 (i.e. for an appropriate enclosing tube 108) and has a longitudinal location that is fixed relative to 211 and 212.

(iv) the contour of cuff 200 that is distal to LDE_TCP(30 cm H₂O) 399 is labelled as 433 FIG. 4A shows both the contour 432 (i.e. relevant when cuff 200 is inflated to 5 cm H₂O) and contour 433 (i.e. relevant when cuff 200 is inflated to 30 cm H2O.

(v) comparing contours 432 and 433 shows the deformation of the neck portion 289 of cuff 200. Clearly, inflation of cuff from 5 cm H₂O to 30 cm H₂O serves to significantly increase the volume within cuff 200. Thus can be observed in FIG. 9—curves 261 and 262. The increase in volume may afford a location for ‘spillover’ gas which enters into cuff 200, (vi) as shown in FIG. 4B, TCP(30 cm H₂O) may be divided into two portions—TCP_{WRINKLED} (30 cm H₂O) which is the portion of cuff 200 in wrinkled contact with enclosing tube 108 and TCP_{UNWRINKLED} (30 cm H₂O) which is the portion of cuff 200 in unwrinkled contact with enclosing tube 108. At least some of TCP_{UNWRINKLED} (30 cm H₂O) may because inflation from 5 cm H₂O to 30 cm H₂O deforms neck portion 289 of cuff 200 to stretch at least a portion of cuff 200 into contact with enclosing tube 108.

(vii) comparing FIG. 3A to FIG. 4A, it is quite clear that the contact length LENGTH(TPC) between cuff 200 and enclosing tube 108 increases as pressure increases, even quite significantly—e.g. due to inflation-driven deformation of neck portion 289.

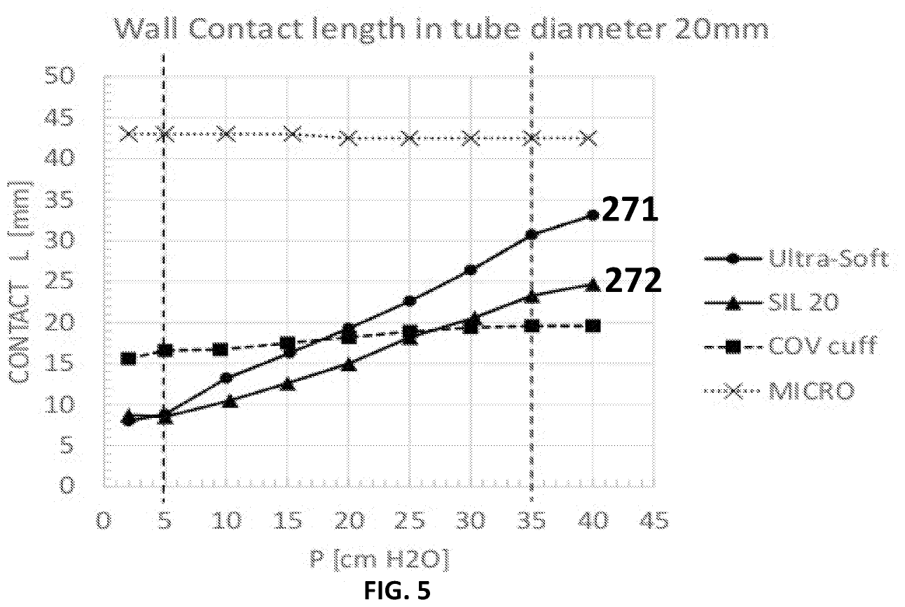

Reference is made to FIG. 5 which shows how contact length LENGTH(TPC) between cuff 200 and enclosing tube 108 increases as pressure increases for a specific example of a straight in-vitro enclosing (i.e. perfectly cylindrical) test tube 108 having a tube diameter (i.e. inner diameter) of 20 mm Curves 271 and 272 refer to embodiments of the invention—in contrast, MICRO and COV cuff (Covidien cuff) are prior art cuffs.

From FIG. 5, it is possible to observe the following:

(i) even at low pressures of at most 5 cm H₂O, the contact is length is non-zero;

(ii) a ratio between the contact length at relatively ‘high pressures’ and the contact length at relatively low pressures' is significantly greater than can be observed in the MICRO or COV cuff prior art cuffs.

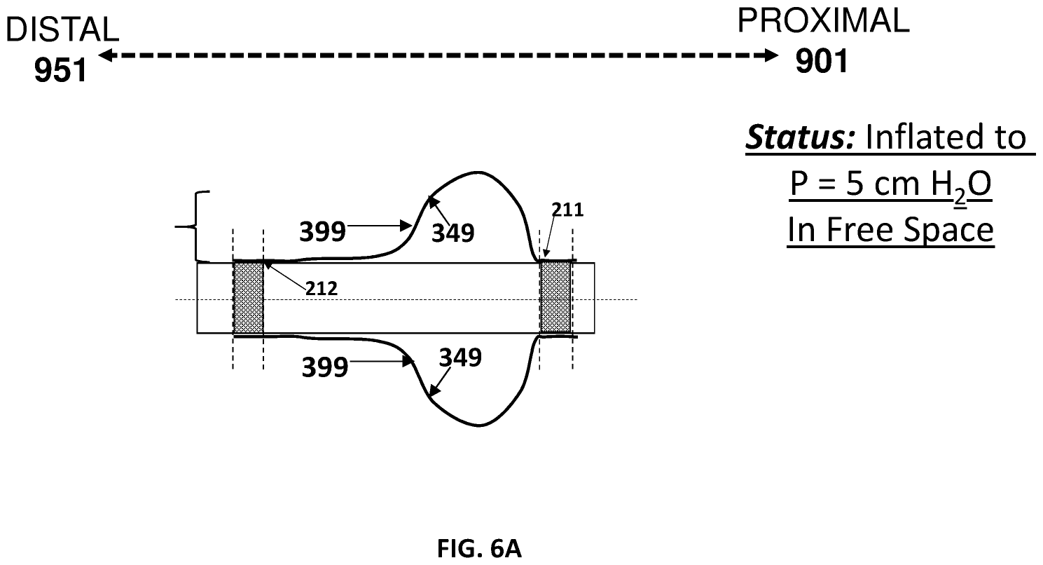

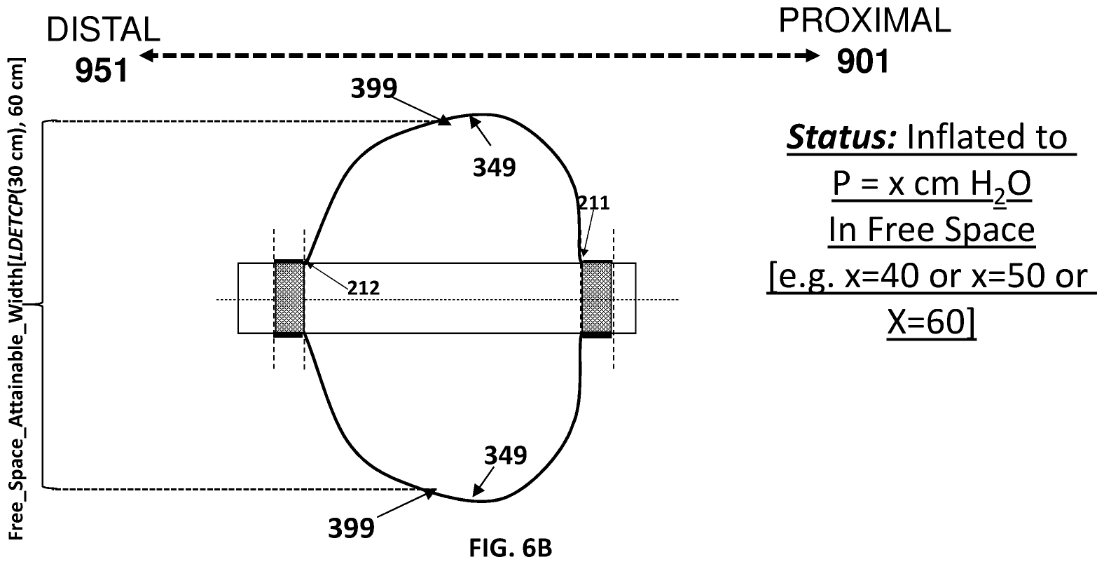

FIG. 6A shows the cuff 200 when it is (i) in free space and (ii) inflated to 5 cm H₂O. FIG. 6B shows the cuff 200 when it is (i) in free space and (ii) inflated to x cm H2O, where x is a ‘larger’ number (e.g. 40 or 50 or 60). As noted above, both 349 and 399 are properties of cuff 200 (i.e. for an appropriate enclosing tube 108) and has a longitudinal location that is fixed relative to 211 and 212.

Thus, comparing FIG. 6B to FIG. 6A, it is shown that the width of cuff 200 at location 399 is significantly larger in FIG. 6B than in FIG. 6A, even as the longitudinal location is fixed.

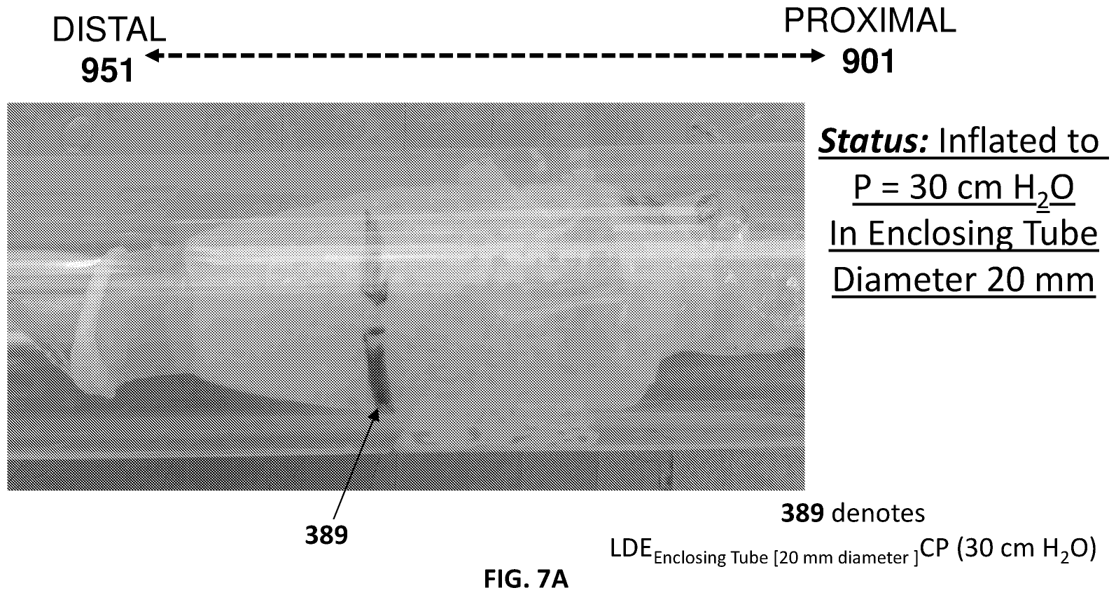

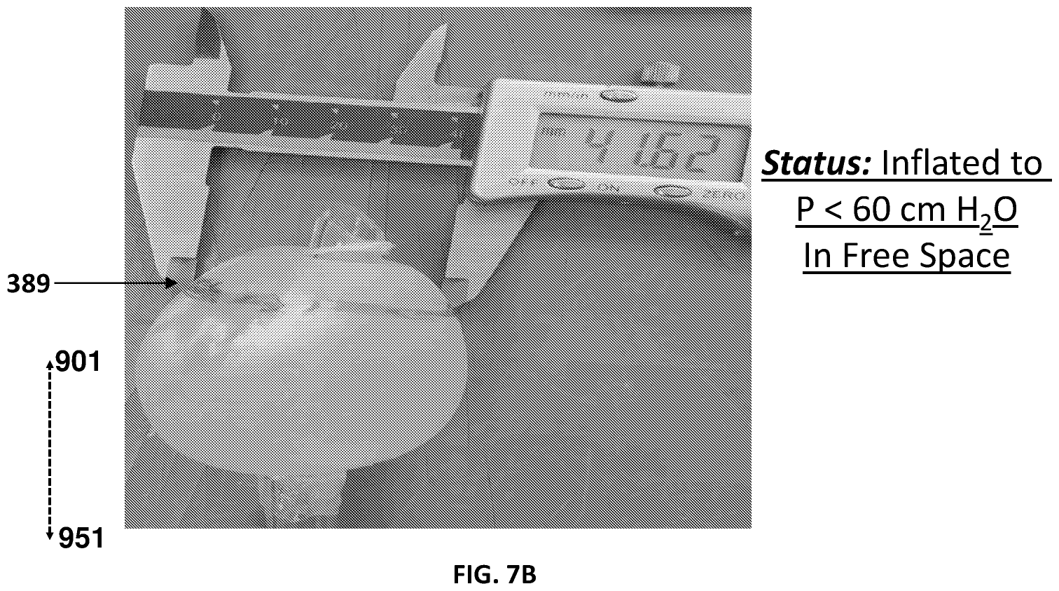

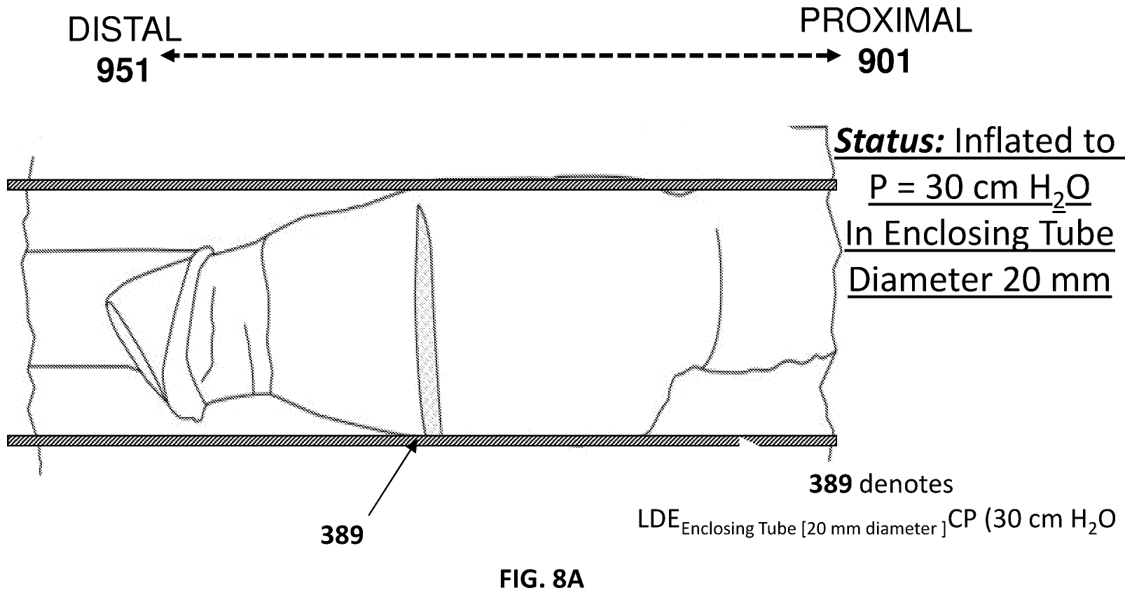

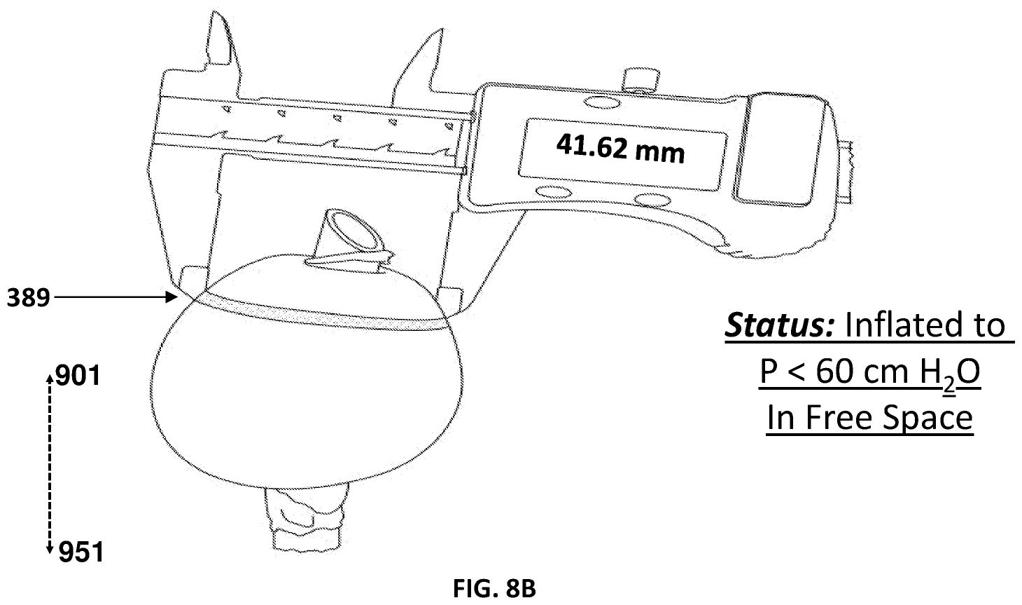

FIG. 7A-7B and 8A-8B relate to an experiment performed where the enclosing tube 108 is a perfectly straight cylindrical enclosing in-vitro test tube having a diameter of 20 mm. First, cuff 200 is inflated (see FIGS. 7A and 8A) to 30 cm H₂O in order to establish the leading distal edge 389 of contact portion TCP of cuff 200 with tube 108 at 30 cm H₂O. Subsequently, cuff 200 is removed from enclosing tube 108—due to the material properties of cuff 200 (e.g. of the neck portion thereof), it is possible to inflate cuff so the width at location 389 (i.e. which was defined in FIGS. 7A and A) increases significantly—according to this experiment, to about 41 mm which in this experiment is about double the width of tube 108.

FIG. 9 illustrates results of an experiment where both prior-art and cuff according to embodiments of the invention are either (i) in free space or (ii) disposed within a perfectly straight cylindrical enclosing in-vitro test tube having a diameter of 20 mm Each of the cuffs are inflated to various pressures. For various pressures, it is possible to measure the volume V of the cuff. The x axis of FIG. 9 is not V but rather VD which is the difference between (i) the volume of the cuff at any particular pressure and (ii) the volume of the cuff when inflated to a particular initiation pressure.

Observing curve 259, we note that for the ultra-soft free embodiment, the inflatable cuff 200 is incapable of being air-inflated to a pressure of 30 cm of H₂O or incapable of being air-inflated to a pressure of 28 of cm H₂O incapable of being air-inflated to a pressure of 25 of H₂O—the maximum of curve 259 is below 25 cm of H2O. Curve 261 relates to the SIL 20 embodiment when in free space. As shown in curve 261 of FIG. 9, after reaching a pressure of 20 cm of water an additional 2 cc or 3 cc or 4 cc or 5 cc or more of air is required in order to reach the pressure of 35 cm of water.

Curve 272 relates to the ultra-soft free embodiment when in the perfectly straight cylindrical enclosing in-vitro test tube 108 having a diameter of 20 mm Curve 262 relates to the SIL 20 embodiment when in the perfectly straight cylindrical enclosing in-vitro test tube 108 having a diameter of 20 mm

### FIGS. 10A-10Cs

Taken from the literature—the skilled artisan will understand how this specifies when a ventilation tube 106 is ‘sized for’ a particular human trachea. When a particular ventilation tube 106 is sized for a trachea (i.e. according to the art), we may say that the human trachea is ‘sized for’ that particular ventilation tube 106.

Ventilation tubes and associated cuffs are sized according to intended human user, as summarized in the tables shown in FIGS. 10A to 10C. For the sake of clarity of presentation, an explicit example embodiments discussion may be done in terms of intended adult male user of assumed trachea size of diameter size of 20 mm and associated ventilation tube size 8.0. Yet, it should be understood that the diameters and lengths would scale proportionally to the ventilation tube diameter. But, the discussed pressure values do not change. For example, the statement “at pressure of 20 cm H₂O there must be a contact between the cuff and the bounding testing tube wall” remains valid when a ventilation tube size 7.0 (with associated attached cuff) is tested with a bounding testing tube of diameter of 17 mm, or a ventilation tube size 6.0 (with associated attached cuff) is tested with a bounding testing tube of diameter of 14 mm

### Additional Discussion

A ventilation device comprising:
a. a ventilation tube 106 having a proximal 102 and distal 107 ends; and
b. an inflatable cuff 200 constructed of an elastic material and mounted around the ventilation tube 106 to define proximal 211 and distal 212 cuff attachment locations which are both fixed on an outer surface of the ventilation tube, the inflatable cuff having a proximal bulge portion 287 and a distal neck portion 289 disposed distal to the bulge portion.

In some embodiments, when the mounted cuff 20 is disposed in free space and is inflated with air, a pressure within the cuff 200 first reaches a free-space-inflation peak pressure (FSPIPP) and then decreases upon further inflation, a value of the FSPIPP being between 12 and 35 cm H₂O.

3. In some embodiments, wherein the value of the FSIPP is at most 30 cm H₂O.

4. In some embodiments, wherein the value of the FSIPP is at most 25 cm H₂O.

5. In some embodiments, wherein the value of the FSIPP is at most 21 cm H₂O.

6. In some embodiments, wherein the value of the FSIPP is at most 19 cm H₂O.

7. In some embodiments, wherein the value of the FSIPP is at least 12 cm H₂O.

8. In some embodiments, wherein the value of the FSIPP is at least 15 cm H₂O.

9. In some embodiments, wherein the value of the FSIPP is at least 17 cm H₂O.

10. In some embodiments, wherein the value of the FSIPP is at least 18 cm H₂O.

11. In some embodiments, wherein a length of the mounted cuff is at least 2 cm, the length being defined as a longitudinal displacement between the proximal 211 and distal 212 cuff attachment locations.

12. In some embodiments, wherein a length of the mounted cuff is at least 2.5 cm, the length being defined as a longitudinal displacement between the proximal 211 and distal 212 cuff attachment locations.

13. In some embodiments, wherein a length of the mounted cuff is at least 3 cm, the length being defined as a longitudinal displacement between the proximal 211 and distal 212 cuff attachment locations.

14. In some embodiments, wherein a length of the mounted cuff is at most 6 cm, the length being defined as a longitudinal displacement between the proximal 211 and distal 212 cuff attachment locations.

15. In some embodiments, wherein a length of the mounted cuff is at most 5 cm, the length being defined as a longitudinal displacement between the proximal 211 and distal 212 cuff attachment locations.

16. In some embodiments, wherein a length of the mounted cuff is at most 4 cm, the length being defined as a longitudinal displacement between the proximal 211 and distal 212 cuff attachment locations.

17. In some embodiments (e.g. see FIG. 6A) wherein (I) when the mounted cuff 20 is disposed in free space, inflation of the cuff with air is incapable of causing an interior of the cuff to reach a pressure of 25 cm H₂O; and (ii) when the mounted cuff 20 is disposed within a straight rigid bounding testing tube (SRBTT_{DIAMETER=20 mm}) that has a diameter of 20 mm and that is co-axial with the ventilation tube, inflation of the cuff with air causes an interior of the cuff to reach a pressure of at least p2 cm H₂O, a value of p2 being equal to at least 30.

18. In some embodiments, wherein a value of p2 is at least 35.

19. In some embodiments (e.g. see FIG. 6A) wherein (I) when the mounted cuff 20 is disposed in free space, inflation of the cuff with air is incapable of causing an interior of the cuff to reach a pressure of 30 cm H₂O; and (ii) when the mounted cuff 20 is disposed within a straight rigid bounding testing tube (SRBTT_{DIAMTER=20 mm}) that has a diameter of 20 mm and that is co-axial with the ventilation tube, inflation of the cuff with air causes an interior of the cuff to reach a pressure of at least p2 cm H₂O, a value of p2 being equal to at least 35.

20. In some embodiments, wherein a value of p2 is at least 40.

21. In some embodiments wherein when the mounted cuff 20 (i) is disposed within a straight rigid bounding testing tube (SRBTT_{DIAMETER=20 mm}) that has a diameter of 20 mm and that is co-axial with the ventilation tube and (ii) is inflated with air to a pressure of 20 cm H₂O, then in order to further inflate the mounted cuff to a pressure of 30 cm H₂O, at least Vol_{ADD} of air must be forced into the cuff, a value of being Vol_{ADD} at least 1 cc.

22. In some embodiments, wherein a value of Vol_{ADD} is at least 2 cc.

23. In some embodiments, wherein a value of Vol_{ADD} is at least 3 cc.

24. In some embodiments, wherein a value of Vol_{ADD} is at least 5 cc.

25. In some embodiments (e.g. see FIG. 6A) when the mounted cuff 20 (i) is disposed within a straight rigid bounding testing tube (SRBTT_{DIAMTER=20 mm}) that has a diameter of 20 mm and that is co-axial with the ventilation tube and (ii) is inflated with air to a pressure of 15 cm H₂O, then in order to further inflate the mounted cuff to a pressure of 25 cm H₂O, at least Vol_{ADD} of air must be forced into the cuff, a value of being Vol_{ADD} at least 1 cc.

26. In some embodiments, wherein a value of Vol_{ADD} is at least 2 cc.

27. In some embodiments, wherein a value of Vol_{ADD} is at least 3 cc.

28. In some embodiments, wherein a value of Vol_{ADD} is at least 5 cc.

29. In some embodiments, wherein throughout a 2 cm-central-portion of the cuff whose longitudinal center is halfway between the proximal 211 and distal 212 cuff attachment locations and whose length is 2 cm, (i) a Shore A value of material of the cuff is at most Shore_A_Max; (ii) a value of Shore_A_Max at most 30.

30. In some embodiments, wherein a value of Shore_A_Max is at most 20.

31. In some embodiments, wherein a value of Shore_A_Max is at most 15.

32. In some embodiments, wherein a value of Shore_A_Max is at most 10.

33. In some embodiments, wherein a value of Shore_A_Max is at most 20.

34. In some embodiments, wherein (i) throughout the 2 cm-central-portion of the cuff, the Shore A value of material of the cuff has a value of at least Shore_A_Min; (ii) a value of Shore_A_Max at least 4.

35. In some embodiments, wherein a value of Shore_A_Min is at least 5.

36. In some embodiments, wherein a value of Shore_A_Min is at least 6.

37. In some embodiments, wherein throughout the 2 cm-central-portion of the cuff, a wall thickness of the cuff is at least 0.1 mm.

38. In some embodiments, wherein throughout the 2 cm-central-portion of the cuff, a wall thickness of the cuff is at least 0.25 mm.

39. In some embodiments, wherein throughout the 2 cm-central-portion of the cuff, a wall thickness of the cuff is at most 1 mm.

40. In some embodiments, wherein throughout the 2 cm-central-portion of the cuff, a wall thickness of the cuff is at most 0.8 mm

41. In some embodiments, wherein the cuff is constructed from at least one of silicone, or Thermoplastic Rubber (TPR) Compounds, or alternatively thermoplastic elastomers (TPE), or combinations thereof.

42. In some embodiments, (e.g. see FIG. 6B) wherein when the mounted cuff 20 is disposed within a straight rigid bounding testing tube (SRBTT_{DIAMTER=20 mm}) that has a diameter of 20 mm and that is co-axial with the ventilation tube and (i) is inflated to 5 cm H₂O, a contact length of the cuff is CL_5, (ii) is inflated to 10 cm H₂O, a contact length of the cuff is CL_10; (iii) is inflated to 15 cm H₂O, a a contact length of the cuff is CL_10; (iii) is inflated to 15 cm H₂O, a contact length of the cuff CL_15; (iii) is inflated to 20 cm H₂O, a contact length is CL_20; (iii) is inflated to 25 cm H₂O, a contact length of the cuff is CL_25; and (iii) is inflated to 30 cm H₂O, a contact length of the cuff CL_30.

44. In some embodiments, wherein a ratio between CL_5 and the length of the cuff LENGTH_{CUFF} is at most 0.1.

45. In some embodiments, wherein a ratio between CL_25 and CL5 is at least 1.5 or at least 2 or at least 3 or at least 5 or at least 10.

46. In some embodiments, (e.g. see FIG. 6B) wherein a ratio between CL_25 and CL10 is at least 1.25.

47. In some embodiments, wherein a ratio between CL_25 and CL10 is at least 1.5.

48. In some embodiments, wherein a ratio between CL_25 and CL10 is at least 1.75.

49. In some embodiments, wherein a ratio between CL_25 and CL10 is at least 2.

50. In some embodiments, wherein a ratio between CL_30 and CL10 is at least 1.5.

51. In some embodiments, wherein a ratio between CL_30 and CL10 is at least 1.75.

52. In some embodiments, wherein a ratio between CL_30 and CL10 is at least 2.

53. In some embodiments, wherein a ratio between CL_30 and CL10 is at least 2.25.

54. In some embodiments, wherein a ratio between CL_30 and CL10 is at least 2.5.

55. In some embodiments, wherein when the mounted cuff 20 is disposed within a straight rigid bounding testing tube (SRBTT_{DIAMTER=20 mm}) that has a diameter of 20 mm and that is co-axial with the ventilation tube, after inflating to a pressure of 15 cm H₂O so that a contact-portion of the cuff (a length of the contact-potion is a contact-length) is in contact with the straight rigid bounding testing tube (SRBTT_{DIAMETER=20 mm}), further inflation of the mounted cuff to a pressure of 30 cm H₂O causes a proximal extreme of the contact-portion to move proximally by prox_15_30, wherein a value of prox_15_30 is positive—for example, a value of prox_15_30 is at least 0.5 mm or at least 1 mm or at least 2 mm and/or at most 5 mm 56. In some embodiments, wherein when the mounted cuff 20 is disposed within a straight rigid bounding testing tube (SRBTT_{DIAMETER=20 mm}) that has a diameter of 20 mm and that is co-axial with the ventilation tube, after inflating to a pressure of 15 cm H₂O so that a contact-portion of the cuff is in contact with the straight rigid bounding testing tube (SRBTT_{DIAMETER=20 mm}), further inflation of the mounted cuff to a pressure of 30 cm H₂O causes a distal extreme of the contact-portion to move distally by dist_15_30, wherein a value of dist_15_30 is positive—for example, a value of dist_15_30 is at least 3 mm or at least 5 mm or at least 7.5 mm or at least 10 mm. 57. In some embodiments, (e.g. this relates to shape-deformation of the cuff when inflated) wherein a ratio between dist_15_30 and prox_15_30 is at least 1.25 or at least 1.5 or at least 3 or at least 5 or at least 10.

58. In some embodiments, wherein when (i) the mounted cuff 20 is disposed within a straight rigid bounding testing tube (SRBTT_{DIAMETER=20 mm}) that has a diameter of 20 mm and that is co-axial with the ventilation tube, and (ii) the mount cuff is inflated to a pressure of 25 cm H₂O, a contact-portion CP(25) of the cuff is in contact with the straight rigid bounding testing tube (SRBTT_{DIAMETER=20 mm}).

59. The ventilation device of claim 58 wherein a ratio between (i) a length of the contact-portion CP(25) which is wrinkle-free and (ii) a length of the contact-portion CP(25) which exhibits wrinkles, at least 1.2 or at least 1.3 or at least 1.4 or at least 1.5 or at least 1.6 or at least 1.8 or at least 2.

61. In some embodiments, wherein:
A. when (i) the mounted cuff 20 is disposed within a straight rigid bounding testing tube (SRBTT_{DIAMTER=20 mm}) that has a diameter of 20 mm and that is co-axial with the ventilation tube, and (ii) the mount cuff is inflated to a pressure of 20 cm H₂O, a wrinkle-free-contact-portion WF_CP(20) of the cuff is both in contact with the straight rigid bounding testing tube (SRBTT_{DIAMETER=20 mm}) is free of wrinkles;
B. upon further inflation to a pressure of 30 cm H2O, a wrinkle-free-contact-portion WF_CP(20) of the cuff is both in contact with the straight rigid bounding testing tube (SRBTT_{DIAMETER=20 mm}) is free of wrinkles;
C. a proximal extreme of WF_CP(30) is located proximal to a proximal extreme of WF_CP(20).

62. In some embodiments, wherein when (i) the mounted cuff 20 is disposed within a straight rigid bounding testing tube (SRBTT_{DIAMETER=20 mm}) that has a diameter of 20 mm and that is co-axial with the ventilation tube, and (ii) the mount cuff is inflated to a pressure of 25 cm H₂OL
i. a contact-portion CP(25) of the cuff is in contact with the straight rigid bounding testing tube (SRBTT_{DIAMETER=20 mm});
ii. at least a portion WF_CP(25) of the contact-portion CP(25) is wrinkle fee.

63. In some embodiments, of wherein another portion WRINKLED_CP(25) of the contact-portion CP(25) is wrinkled, and wherein a ratio between (i) a length of WF_CP(25) and (ii) a length of

WRINKLED_CP(25) is at least 1.2 or at least 1.4 or at least 1.5 or at least 1.6 or at least 1.8 or at least 2 or at least 2.5 or at least 3 or at least 5.

64. In some embodiments, wherein a most-distal-contact-at-25 cm location of the ventilation tube is defined as follows:
A. when (i) the mounted cuff 20 is disposed within a straight rigid bounding testing tube (SRBTT_{DIAMETER=20 mm}) that has a diameter of 20 mm and that is co-axial with the ventilation tube, and (ii) the mount cuff is inflated to a pressure of 25 cm H₂O, a contact-portion CP(25) of the cuff is in contact with the straight rigid bounding testing tube (SRBTT_{DIAMETER=20 mm});
B. the most-distal-contact-at-25 cm location of the ventilation tube is defined as a proximal extreme of the contact-portion CP(25).

65. In some embodiments, wherein the most-distal-contact-at-25 cm is located in the proximal half (e.g. in the proximal third) of the cuff.

66. In some embodiments, wherein when the mounted cuff 20 is in free space, the cuff is inflatable so that a width of the cuff CUFF_WIDTH(most-distal-contact-at-25 cm) at a location on the cuff longitudinally corresponding to the most-distal-contact-at-25 cm location of the ventilation tube is at least 22 mm or at least 25 mm or at least 30 mm or at least 35 mm or at least 40 mm

67. In some embodiments, wherein when the mounted cuff 20 is in free space, all locations in the central 30% of the cuff are inflatable to a width of at least 22 mm or at least 25 mm or at least 30 mm or at least 35 mm or at least 40 mm.

68. In some embodiments, wherein when the mounted cuff 20 is in free space, all locations in the central 50% of the cuff are inflatable to a width of at least 22 mm or at least 25 mm or at least 30 mm or at least 35 mm or at least 40 mm.

69. In some embodiments, wherein when the mounted cuff 20 is in free space, all locations in the central 7-% of the cuff are inflatable to a width of at least 22 mm or at least 25 mm or at least 30 mm or at least 35 mm or at least 40 mm.

Another discussion of how inflation of the cuff induces deformation/change of the cuff shape is now provided.

70. In some embodiments, wherein the mounted cuff 20 is geometrically dividable by length to four equal portions that are (i) a most distal 25% MD_25; (ii) a second most distal 25% SMD_25; (iii) a second most proximal 25% SMP_25; and (iv) a most proximal 25% MP_25. (e.g. see FIGS. 3A-3B)

71. In some embodiments, wherein when the mounted cuff 20 is in free space and inflated to a pressure of 5 H₂O, (i) an average width of the mounted cuff over the most distal portion is WIDTH AVG(MD_25,5,free space); (ii) an average width of the mounted cuff over the most second distal portion is WIDTH_AVG(SMD_25,5,free space); (iii) an average width of the mounted cuff over the most second proximal portion is WIDTH_AVG(SMP_25,5,free space); and (iv) an average width of the mounted cuff over the most proximal portion is WIDTH AVG(MP_25,5,free space).

72. In some embodiments, wherein when the mounted cuff 20 is in free space and inflated to a pressure of 26 H₂O, (i) an average width of the mounted cuff over the most distal portion is WIDTH AVG(MD_25,26,free space); (ii) an average width of the mounted cuff over the most second distal portion is WIDTH AVG(SMD_25,26,free space); (iii) an average width of the mounted cuff over the most second proximal portion is WIDTH_AVG(SMP_25,26,free space); and (iv) an average width of the mounted cuff over the most proximal portion is WIDTH AVG(MP_25,26,free space).

73. In some embodiments, wherein a ratio between WIDTH_AVG(MD_25,26,free space) and WIDTH AVG(MD_25,5,free space) is at least 1.5 or at least 2 or at least 3. This may relate to neck ‘lift-off’ when the cuff is inflated.

74. The ventilation device of any one of claims 72-73 wherein a ratio between WIDTH AVG(SMD_25,26,free space) and WIDTH AVG(SMD_25,5,free space) is at least 1.5 or at least 2 or at least 2.5 or at least 3. This may relate to neck ‘lift- off’ when the cuff is inflated—e.g. significant lift-off in a distal 25% of the cuff.

75. In some embodiments, wherein a ratio between (i) a ratio between WIDTH AVG(SMD_25,26,free space) and WIDTH_AVG(SMD_25,5,free space) and (ii) a ratio between WIDTH AVG(SMP_25,26,free space) and WIDTH AVG(SMP_25,5,free space) is at least 1.2 or at least 1.4 or at least 1.6 or at least 1.8 or at least 2.

76. In some embodiments, wherein a ratio between (i) a ratio between WIDTH AVG(SMD_25,26,free space) and WIDTH_AVG(SMD_25,5,free space) and (ii) a ratio between WIDTH AVG(MP_25,26,free space) and WIDTH_AVG(MP_25,5,free space) is at least 1.2 or at least 1.4 or at least 1.6 or at least 1.8 or at least 2.

77. In some embodiments, wherein the cuff further has most-proximal and most-distal half-height geometric-locations whose position relative to the ventilation tube varies as a function of inflation pressure of the inflatable cuff.

78. In some embodiments, wherein the cuff outer diameter is measured along an axis that is substantially orthogonal to the axis of the ventilation tube.

79. In some embodiments, wherein when the cuff is fully inflated with air in free space so that the cuff is inflated while unbounded and not inserted in a patient trachea, to and beyond 5 cm H₂O initiation pressure:
A. a volume of the cuff at a 5 cm H₂O initiation pressure is V1 and its outer cuff diameter at the widest location is CDS.
B. a pressure within the cuff reaches a free-space-inflation peak pressure PPFS whose value is between 18 and 35 cm H₂O;
C. at the free-space-inflation peak pressure PPFS a volume of the cuff is VP;
D. upon further inflation a pressure decreases after reaching the free-space-inflation peak pressure PPFS; and
E. when the cuff is inflated from 5 cm H₂O to peak pressure PPFS, a ratio between an axial displacement of the most-distal half-height location and an axial displacement of the most-proximal half-height location is at least two.

80. In some embodiments, wherein when the cuff placed within a straight rigid bounding testing tube (SRBTT_{DIAMETER=20 mm}) that has a diameter of 20 mm and that is co-axial with the ventilation tube, inflation of the cuff to a pressure of 30 cm H2O, forms a wrinkle-free band of length LF against the testing tube wall, such that at least 50% or at least 75% or at least 85% or at least 90% of the cuff contact length with testing tube wall is wrinkle-free.

81. In some embodiments, wherein when the cuff placed within a straight rigid bounding testing tube of a diameter that is larger than CD5 by 1mm and that is co-axial with the ventilation tube, and when the cuff is inflated to a volume V3 that is less than VP, the pressure within the cuff exceeds the free-space-inflation peak pressure PPFS by at least 5 cm H₂O.

82. In some embodiments, wherein a length of the wrinkle-free band is at least 5 mm

83. In some embodiments, wherein a length of the wrinkle-free band is at least 10 mm

84. In some embodiments, wherein when the cuff placed within a straight rigid bounding testing tube (SRBTT_{DIAMETER=20 mm}) that has a diameter of 20 mm and that is co-axial with the ventilation tube, and the cuff is inflated to a pressure of 30 cm H2O, the diameter of the cuff at a majority of points along the wrinkled free band is smaller by at least 5% than when the cuff is inflated with air in unbounded free space, to a pressure of 30 cm H2O or to the free-space-inflation peak pressure PPFS less than 30 cm H2O.

85. In some embodiments, wherein the proximal bulge portion 201 and the distal neck portion 202 are such that
a. the proximal bulge portion 201 is extending from the most-distal half-height location 204 proximally up to the proximal attachment 211 of the cuff to the tube 106;
b. the distal neck portion 202 is extending from the most-distal half-height location 204 distally up to the distal attachment 212 of the cuff to the tube 106;
c. the proximal bulge portion 201 is convex in the cuff section between the most-distal half-height location 204 and most-proximal half-height location 205.

86. In some embodiments, wherein, the distal neck portion 202 is concave, in the sense that a tangent sphere of finite radius is tangent to the cuff at two non-attached locations, a distal tangent point at a distal location on the cuff and a proximal tangent point at a location on the cuff more proximal than the distal tangent point; such that when held in free space,
d. at initiation pressure P1 of 5 cm H₂O the largest tangent sphere has a radius R1, at mid-pressure P2 equal to 15 cm H₂O the largest tangent sphere has a radius R2, at a mid-pressure P3 higher than P2 , P3>P2, the largest tangent sphere has a radius R3; and
e. R1>R3>R3.

87. In some embodiments, wherein, the cuff having most-proximal and most-distal half-height locations whose position relative to the ventilation tube varies as a function of inflation pressure of the inflatable cuff such that: when inflated inside a testing tube of 20 mm diameter, the axial distance between locations of the half-height distal cuff edge 234 at pressure P1 of 5 cmH2O, and half-height distal cuff edge 236 at pressure P5 of 35 cm H₂O is greater than the axial distance, when inflated in free space, between locations of the half-height distal cuff edge 224 at pressure P1 of 5 cmH2O and the half-height distal cuff edge 226 at free-space peak pressure PPFS, by at least 30%, or by at least 50%.

88. In some embodiments, wherein when a cuff 200 is inflated within the testing tube 108, there is a central holding position such that: (a) there is no contact between the cuff and the bounding testing tube wall at initiation pressure of 5 cm H2O; and (b) at pressure of 30 cm H₂O there must be a contact between the cuff and the bounding testing tube wall.

89. In some embodiments, wherein when a cuff 200 is inflated within the testing tube 108, there is a central holding position at which as inflation pressure grows, the cuff contact section 240 length grows such that: (a) at a pressure P4 of 20 cm H2O, having a contact section 240 of axial length L20 greater than 2 mm between the cuff and the testing tube wall, there is distance D2 between distal cuff attachment location 215 and most-distal contact location of the cuff with the bounding tube wall; and (b) at a pressure of 40 cm H2O, having a contact section 240 of axial length L40 greater that 5 mm between the cuff and the testing tube wall, there is distance D3 between distal cuff attachment location 215 and most-distal contact location of the cuff with the bounding tube wall, and (c) L40 is greater than L20 by at least 25%.

90. In some embodiments, wherein at an inflation pressure of 15 cm H2O, the cuff is having a contact section 240 of axial length that is less than 30% of the distance between the proximal cuff attachment 211 and the distal cuff attachment 212.
91. In some embodiments, wherein the cuff 200 is attached to the ventilation tube 106 at a proximal cuff attachment 211 and at a distal cuff attachment 212, where the attachment is in the form of glue or welding or elastic compression with a material of shore value at least twice larger than the shore value of the cuff bulge wall material.

92. In some embodiments, wherein when the mounted cuff is disposed in free space and inflated with air to 5 cm H2O, further inflation of the mounted cuff to the FSPIPP:
1) axially displaces the most distal half-max-elevation location in the distal direction by a first displacement; and
(2) axially displaces the most proximal half-max-elevation location in the proximal direction by a second displacement; and
B. a ratio between the first and second displacements is at least two or at least 3.

93. In some embodiments, wherein the cuff is single-layered.

94. In some embodiments, wherein the ventilation tube is selected from the group consisting of a endotracheal tube (ETT), tracheostomy tube; and a multi-cuff (e.g. dual cuff) laryngeal device tube (e.g. the cuff is a proximal cuff around the multi-cuff tube).

95. In some embodiments, wherein an outside diameter of the ventilation tube is at least 6 mm.

96. In some embodiments, wherein an outside diameter of the ventilation tube is at least 7 mm.

97. In some embodiments, wherein an outside diameter of the ventilation tube is at least 8 mm.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A ventilation device comprising:
a. a ventilation tube having proximal and distal ends; and
b. an inflatable cuff having a proximal bulge portion and a distal neck portion disposed distal to the bulge portion, the inflatable cuff being mounted around the ventilation tube to define proximal and distal cuff attachment locations which are both fixed on an outer surface of the ventilation tube, wherein:
(i) when the tube-mounted cuff is deployed within a human trachea sized for the ventilation tube so that (A) the ventilation tube is co-axial with the human trachea and (B) the tube-mounted cuff is uninflated or inflated to a pressure of 5 cm H2O, a widest portion of the bulge portion of the mounted cuff is in wrinkled contact with the trachea; and
(i) when the tube-mounted cuff is deployed within a human trachea sized for the ventilation tube so that (A) the ventilation tube is co-axial with the human trachea and (B) the tube-mounted cuff is uninflated or inflated to a pressure of 25 cm H2O, a widest portion of the bulge portion of the mounted cuff is in wrinkled contact with the trachea.

2. The device of claim 1 wherein the ventilation tube is sized for an adult human trachea.

3. The ventilation device of any preceding claim wherein a length of the inflatable cuff is at least 2 cm.

4. The ventilation device of any preceding claim wherein the length of the inflatable cuff is at most 6 cm.

5. The device of any preceding claim wherein inflation of the human-trachea-deployed and tube-mounted cuff (i.e. so the ventilation tube is coaxial with the trachea) to a pressure of x cm H₂O defines a trachea-contact portion TCP(x) of the cuff and a leading distal edge LDE_TCP(x) thereof.

6. The ventilation device of claim 5 wherein when the ventilation device is in free space, upon inflation of the inflatable cuff to a pressure of at most 60 cm H₂O, a width of the inflatable cuff at the LDE(TCP(25 cm H₂O)) reaches at least 40 mm and/or at least 1.5 times the width of the human trachea sized for the ventilation tube.

7. The ventilation device of claim 5 wherein when the ventilation device is in free space, upon inflation of the inflatable cuff to a pressure of at most 40 cm H₂O, a width of the inflatable cuff at the LDE(TCP(25 cm H₂O)) reaches at least 40 mm and/or at least 1.5 times the width of the human trachea sized for the ventilation tube.

8. The ventilation device of claim 5 wherein when the ventilation device is in free space, upon inflation of the inflatable cuff to a pressure of at most 60 cm H₂O, a width of the inflatable cuff at the LDE(TCP(30 cm H₂O)) reaches at least 40 mm and/or at least 1.5 times the width of the human trachea sized for the ventilation tube.

9. The ventilation device of claim 5 wherein when the ventilation device is in free space, upon inflation of the inflatable cuff to a pressure of at most 40 cm H₂O 50, a width of the inflatable cuff at the LDE(TCP(30 cm H₂O)) reaches at least 40 mm and/or at least 1.5 times the width of the human trachea sized for the ventilation tube.

10. The ventilation device of any one of claims 5-9 wherein the neck portion is sufficiently deformable such that a ratio between a length of TCP(35 cm) and a length of TCP(5 cm) is at least 1.5 or at least 1.75 or at least 2 or at least 2.25 or at least 2.5 or at least 2.75 or at least 3.

11. The ventilation device of any proceeding claim wherein when the tube-mounted cuff is deployed coaxially within the ventilation-tube-sized human trachea and is inflated to a pressure of 5 cm H₂O, a ratio between:
(i) a length of a band of the cuff in wrinkle-free contact with the ventilation-tube-sized human trachea [defined as zero if none of the cuff contact perimeter [perimeter line of a cut perpendicular to the cuff center axis] is in wrinkle-free contact with the ventilation-tube-sized human trachea]; and
(ii) a length of a band of the cuff in wrinkled contact with the ventilation-tube-sized human trachea is defined as Contact_Length_Ratio[Wrinkle-free:Wrinkeed](5 cm H₂O).

12. The ventilation device of claim 12 wherein a value of Contact_Length_Ratio[Wrinkle-free:Wrinkeed](5 cm H₂O) is at most 0.01 or at most 0.1 or at most 0.2

13. The ventilation device of any proceeding claim wherein when the tube-mounted cuff is deployed within the ventilation-tube-sized human trachea and is inflated to a pressure of 25 cm H₂O, a ratio between:
(i) a length of the cuff in in wrinkle-free contact with the ventilation-tube-sized human trachea [defined as zero if none of the cuff in in wrinkle-free contact with the ventilation-tube-sized human trachea]; and
(ii) a length of the cuff in in wrinkled contact with the ventilation-tube-sized human trachea is defined as Contact_Length_Ratio[Wrinkle-free:Wrinkeed](25 cm H₂O).

14. The ventilation device of claim 13 wherein a value of Contact_Length_Ratio[Wrinkle-free:Wrinkeed](25 cm H₂O) is at least 0.3 or at least 0.5 or at least 1.

15. The ventilation device of any preceding claim wherein: (i) a fraction {i.e. between 0 and 1} of TCP(5 cm) that is wrinkle-free is defined as Fract_Wrinkle-free[TCP(5 cm)]; (ii)) a fraction {i.e. between 0 and 1} of TCP(25 cm) that is wrinkle-free defined as Fract_Wrinkle-free[TCP(25 cm)], and wherein a ratio between Fract_Wrinkle-free[TCP(25 cm)]and—Fract_Wrinkle-free[TCP(5 cm)] is at least 2 or at least 5 or at least 10.

16. The ventilation device of any preceding claim wherein when the ventilation device is in free space, the inflatable cuff furthermore is incapable of being air-inflated to a pressure of 30 cm of H₂O.

17. The ventilation device of any one of claims 1-15 wherein when the ventilation device is in free space, the inflatable cuff furthermore is capable of being air-inflated to a pressure of 35 cm of water such that during a pressure ramp-up, after reaching a pressure of 20 cm of water an additional 4 cc (or an additional 5 cc or more) or more of air is required in order to reach the pressure of 35 cm of water.

18. A method of treating a human patient having a human trachea, the method comprising:
a. providing a ventilation device comprising:
i. a ventilation tube having proximal and distal ends; and
ii. an inflatable cuff having a proximal bulge portion and a distal neck portion disposed distal to the bulge portion, the inflatable cuff being mounted around the ventilation tube to define proximal and distal cuff attachment locations which are both fixed on an outer surface of the ventilation tube, wherein:
b. deploying at least a portion of the ventilation tube within the human trachea of the patient so that the inflatable cuff is deployed therein so that: (A) the cuff is uninflated or inflated to a pressure of 5 cm H₂O and (B) a widest portion of the bulge portion of the mounted cuff is in wrinkled contact with the trachea.

19. The method of claim 18 further comprising inflating the cuff to a pressure of 25 cm H₂O so that a widest portion of the bulge portion of the mounted cuff is in wrinkled contact with the trachea.

20. The method of any of claims 18-19 wherein a length of the inflatable cuff is at least 2 cm.

21. The method of any of claims 18-20 wherein the length of the inflatable cuff is at most 6 cm.

22. The method of any of claims 18-21 further comprising inflating the tube to a pressure of x cm H₂O so to define a trachea-contact portion TCP(x) of the cuff and a leading distal edge LDE_TCP(x) thereof.

23. The method of claim 22 wherein a value of x is 25, and when the ventilation device is in free space, upon inflation of the inflatable cuff to a pressure of at most 60 cm H₂O, a width of the inflatable cuff at the LDE(TCP(25 cm H₂O)) reaches at least 40 mm and/or at least 1.5 times the width of the trachea to which the device is deployed.

24. The method of claim 22 wherein a value of x is 25, and when the ventilation device is in free space, upon inflation of the inflatable cuff to a pressure of at most 40 cm H₂O, a width of the inflatable cuff at the LDE(TCP(25 cm H₂O)) reaches at least 40 mm and/or at least 1.5 times the width of the trachea to which the device is deployed.

25. The method of any one of claims 18-24 wherein further comprising inflating the tube to a pressure of x cm H₂O so to define a trachea-contact portion TCP(x) of the cuff and a leading distal edge LDE_TCP(x) thereof, and the neck portion is sufficiently deformable such that a ratio between a length of TCP(35 cm) and a length of TCP(5 cm) is at least 1.5 or at least 1.75 or at least 2 or at least 2.25 or at least 2.5 or at least 2.75 or at least 3.

26. The method of any proceeding claim wherein when the tube-mounted cuff is deployed coaxially within the ventilation-tube-sized human trachea and is inflated to a pressure of 5 cm H₂O, a ratio between:
(i) a length of a band of the cuff in wrinkle-free contact with the ventilation-tube-sized human trachea [defined as zero if none of the cuff contact perimeter [perimeter line of a cut perpendicular to the cuff center axis] is in wrinkle-free contact with the ventilation-tube-sized human trachea]; and
(ii) a length of a band of the cuff in wrinkled contact with the ventilation-tube-sized human trachea is defined as Contact_Length_Ratio[Wrinkle-free:Wrinkeed](5 cm H₂O).

27. The method claim 26 wherein a value of Contact_Length_Ratio[Wrinkle-free:Wrinkeed](5 cm H₂O) is at most 0.01 or at most 0.1 or at most 0.2

28. The method of any proceeding claim wherein when the tube-mounted cuff is deployed within the ventilation-tube-sized human trachea and is inflated to a pressure of 25 cm H₂O, a ratio between:
(i) a length of the cuff in in wrinkle-free contact with the ventilation-tube-sized human trachea [defined as zero if none of the cuff in in wrinkle-free contact with the ventilation-tube-sized human trachea]; and
(ii) a length of the cuff in in wrinkled contact with the ventilation-tube-sized human trachea is defined as Contact_Length_Ratio[Wrinkle-free:Wrinkeed](25 cm H₂O).

29. The method of claim 13 wherein a value of Contact_Length_Ratio[Wrinkle-free: Wrinkeed](25 cm H₂O) is at least 0.3 or at least 0.5 or at least 1.

30. The method of any preceding claim wherein: (i) a fraction {i.e. between 0 and 1} of TCP(5 cm) that is wrinkle-free is defined as Fract_Wrinkle-free[TCP(5 cm)]; (ii)) a fraction {i.e. between 0 and 1} of TCP(25 cm) that is wrinkle-free defined as Fract_Wrinkle-free[TCP(25 cm)], and wherein a ratio between Fract_Wrinkle-free[TCP(25 cm)] and—Fract_Wrinkle-free[TCP(5 cm)] is at least 2 or at least 5 or at least 10.

31. The method of any of claims 18-30 wherein when the ventilation device is in free space, the inflatable cuff furthermore is incapable of being air-inflated to a pressure of 30 cm of H₂O.

32. The method of any of claims 18-30 wherein when the ventilation device is in free space, the inflatable cuff furthermore is capable of being air-inflated to a pressure of 35 cm of water such that during a pressure ramp-up, after reaching a pressure of 20 cm of water an additional 4 cc or more, or an additional 5 cc or more of air is required in order to reach the pressure of 35 cm of water.

33. A ventilation device comprising:
a. a ventilation tube having proximal and distal ends; and
b. an inflatable cuff having a proximal bulge portion and a distal neck portion disposed distal to the bulge portion, the inflatable cuff being mounted around the ventilation tube to define proximal and distal cuff attachment locations which are both fixed on an outer surface of the ventilation tube, wherein:
(i) when the tube-mounted cuff is deployed within a rigid in-vitro enclosing tube having an inner diameter between 18 mm and 21 mm so that (A) the ventilation tube is co-axial with the rigid in-vitro enclosing tube and (B) the tube-mounted cuff is uninflated or inflated to a pressure of 5 cm H2O, a widest portion of the bulge portion of the mounted cuff is in wrinkled contact with an inner wall of the rigid in-vitro enclosing tube and
(i) when the tube-mounted cuff is deployed within a rigid in-vitro enclosing tube having an inner diameter between 18 mm and 21 mm so that (A) the ventilation tube is co-axial with the rigid in-vitro enclosing tube and (B) the tube-mounted cuff is uninflated or inflated to a pressure of 25 cm H2O, a widest portion of the bulge portion of the mounted cuff is in wrinkled contact with an inner wall of the rigid in-vitro enclosing tube.

34. The device of claim 33 wherein the ventilation tube is sized for an adult human trachea.

35. The ventilation device of any preceding claim wherein a length of the inflatable cuff is at least 2 cm.

36. The ventilation device of any preceding claim wherein the length of the inflatable cuff is at most 6 cm.

37. The device of any preceding claim wherein inflation of the rigid-enclosing-tube-deployed and tube-mounted cuff (i.e. so the ventilation tube is coaxial with the rigid in-vitro enclosing tube) to a pressure of x cm H₂O defines a enclosing-tube-contact portion ETCP(x) of the cuff and a leading distal edge LDE_[(]ETCP(x) thereof.

38. The ventilation device of claim 37 wherein when the ventilation device is in free space, upon inflation of the inflatable cuff to a pressure of at most 60 cm H₂O, a width of the inflatable cuff at the LDE(ETCP(25 cm H₂O)) reaches at least 40 mm and/or at least 1.5 times the internal width of the rigid in-vitro enclosing tube.

39. The ventilation device of claim 37 wherein when the ventilation device is in free space, upon inflation of the inflatable cuff to a pressure of at most 40 cm H₂O, a width of the inflatable cuff at the LDE(ETCP(25 cm H₂O)) reaches at least 40 mm and/or at least 1.5 times the internal width of the rigid in-vitro enclosing tube.

40. The ventilation device of claim 37 wherein when the ventilation device is in free space, upon inflation of the inflatable cuff to a pressure of at most 50 cm H₂O, a width of the inflatable cuff at the LDE(ETCP(30 cm H₂O)) reaches at least 40 mm and/or at least 1.5 times the internal width of the rigid in-vitro enclosing tube.

41. The ventilation device of claim 37 wherein when the ventilation device is in free space, upon inflation of the inflatable cuff to a pressure of at most 40 cm H₂O, a width of the inflatable cuff at the LDE(ETCP(30 cm H₂O)) reaches at least 40 mm and/or at least 1.5 times the internal width of the rigid in-vitro enclosing tube and/or wherein the neck portion is sufficiently deformable such that a ratio between a length of ETCP(35 cm) and a length of ETCP(5 cm) is at least 1.5 or at least 1.75 or at least 2 or at least 2.25 or at least 2.5 or at least 2.75 or at least 3.

42. The ventilation device of any proceeding claim wherein when the tube-mounted cuff is deployed coaxially within the in vitro enclosing tube and is inflated to a pressure of 5 cm H₂O, a ratio between:
(i) a length of a band of the cuff in wrinkle-free contact with the in vitro enclosing tube [defined as zero if none of the cuff contact perimeter [perimeter line of a cut perpendicular to the cuff center axis] is in wrinkle-free contact with the in vitro enclosing tube]; and
(ii) a length of a band of the cuff in wrinkled contact with the in vitro enclosing tube is defined as Contact_Length_Ratio[Wrinkle-free:Wrinkeed](5 cm H₂O).

43. The ventilation device of claim 42 wherein a value of Contact_Length_Ratio[Wrinkle-free:Wrinkeed](5 cm H₂O) is at most 0.01 or at most 0.1 or at most 0.2.

44. The ventilation device of any proceeding claim wherein when the tube-mounted cuff is deployed within the in vitro enclosing tube and is inflated to a pressure of 25 cm H₂O, a ratio between:
(i) a length of the cuff in in wrinkle-free contact with the in vitro enclosing tube [defined as zero if none of the cuff in in wrinkle-free contact with the in vitro enclosing tube]; and
(ii) a length of the cuff in in wrinkled contact with the in vitro enclosing tube is defined as Contact_Length_Ratio[Wrinkle-free:Wrinkeed](25 cm H₂O).

46. The ventilation device of claim 45 wherein a value of Contact_Length_Ratio[Wrinkle-free:Wrinkeed](25 cm H₂O) is at least 0.3 or at least 0.5 or at least 1.

47. The ventilation device of any preceding claim wherein: (i) a fraction {i.e. between 0 and 1} of TCP(5 cm) that is wrinkle-free is defined as Fract_Wrinkle-free[TCP(5 cm)]; (ii)) a fraction {i.e. between 0 and 1} of TCP(25 cm) that is wrinkle-free defined as Fract_Wrinkle-free[TCP(25 cm)], and wherein a ratio between Fract_Wrinkle-free[TCP(25 cm)] and—Fract_Wrinkle-free[TCP(5 cm)] is at least 2 or at least 5 or at least 10.

48. The ventilation device of any one of claims 33-47 wherein when the ventilation device is in free space, the inflatable cuff furthermore is incapable of being air-inflated to a pressure of 30 cm of H₂O.

49. The ventilation device of any one of claims 33-47 wherein when the ventilation device is in free space, the inflatable cuff furthermore is capable of being air-inflated to a pressure of 35 cm of water such that during a pressure ramp-up, after reaching a pressure of 20 cm of water an additional 4 cc or 5 cc or more of air is required in order to reach the pressure of 35 cm of water.

50. The ventilation device of any one of claims 33-47 wherein when the ventilation device is in free space, the inflatable cuff furthermore is capable of being air-inflated to a pressure of 35 cm of water such that during a pressure ramp-up, after reaching a pressure of 20 cm of water an additional 4 cc or 5 cc or more of air is required in order to reach the pressure of 35 cm of water.

51. The device of any preceding claim, wherein throughout a 2 cm-central-portion of the cuff whose longitudinal center is halfway between the proximal and distal cuff attachment locations and whose length is 2 cm, (i) a Shore A value of material of the cuff is at most Shore_A_Max; (ii) a value of Shore_A_Max at most 30.

52. The device of any preceding claim, wherein a value of Shore_A_Max is at most 20 or at most 15 or at most 10.

53. The device of any preceding claim wherein (i) throughout the 2 cm-central-portion of the cuff, the Shore A value of material of the cuff has a value of at least Shore_A_Min; (ii) a value of Shore_A_Max at least 4 or least 5.

54. The device of any preceding claim wherein throughout the 2 cm-central-portion of the cuff, a wall thickness of the cuff is at least 0.1 mm or at least 0.25 or at least 1 mm.

55. The device of any preceding claim herein throughout the 2 cm-central-portion of the cuff, a wall thickness of the cuff is at most 0.8 mm.

56. The ventilation device of any preceding claim wherein the cuff is constructed from at least one of silicone, or Thermoplastic Rubber (TPR) Compounds, or alternatively thermoplastic elastomers (TPE), or combinations thereof.
